(19) United States
(12) Patent Application Publication
(10) Pub. No.: US 2025/0011371 A1
(43) Pub. Date: Jan. 9, 2025

(54) RECOMBINANT SARS-CoV-2 IMMUNOGENIC PROTEIN PRODUCED IN PLANTS AND THE USE THEREOF

(71) Applicant: BAIYA PHYTOPHARM CO., LTD., Bangkok (TH)

(72) Inventor: Waranyoo PHOOLCHAROEN, Bangkok (TH)

(21) Appl. No.: 18/276,605

(22) PCT Filed: Mar. 19, 2021
(86) PCT No.: PCT/TH2021/000009

§ 371 Date: Aug. 9, 2023

(51) Int. Cl. C07K 14/005; A61K 39/215; A61P 31/14; C12N 15/82

(52) U.S. Cl. CPC C07K 14/005; A61K 39/215; A61P 31/14; C12N 15/8258; A61K 2039/54; A61K 2039/545; C07K 2319/30; C12N 2770/20022

(57) ABSTRACT

The present invention demonstrates a recombinant vector for producing immunogenic substance from plants which can induce an immune response in mammals against the coronavirus disease 2019 (COVID-19). Said recombinant vector comprises at least a fragment of SARS CoV-2 receptor binding domain protein (SARS CoV-2 RBD) and a fusion protein sequence. The recombinant vector is introduced into plant cells, preferably by means of Agrobacterium sp., thereby the plant cell can express a recombinant protein which can act as an immunogenic substance. The recombinant protein of the present invention significantly demonstrates an ability to trigger immunogenicity in mammals which prevents infectious disease caused by severe acute respiratory syndrome coronavirus 2. Further, the method of inducing an immune response against SARS-CoV-2 in mammals is also provided herein. The present invention further demonstrates the use of such recombinant protein as a vaccine to prevent the coronavirus disease 2019 (COVID-19).

### REFERENCE TO SEQUENCE LISTING

This application is being filed electronically and includes an electronically submitted sequence listing in .txt format. The .txt file contains a sequence listing entitled “Sequence Listing—10139985-51132644_ST25.txt” created on May 16, 2024 and is 32,282_bytes in size. The sequence listing contained in this .txt file is part of the specification and is hereby incorporated by reference herein in its entirety.

### FIELD OF INVENTION

This invention relates to a recombinant SARS-CoV-2 immunogenic protein produced in plants and the use thereof

### BACKGROUND OF INVENTION

In late December 2019, a new infectious disease caused by a novel strain of coronavirus was first reported in Wuhan, the capital of Hubei province, China. The World Health Organization (WHO) announced the official name for this disease as the coronavirus disease (COVID-19), which is caused by severe acute respiratory syndrome coronavirus 2 (SARS-CoV-2), and declared the outbreak of a pandemic thereafter. SARS-CoV-2 is genetically related to the coronavirus which causes severe acute respiratory syndrome (SARS) of which there was an outbreak in 2003. A number of experts have traced back the coronovirus and discovered that COVID-19 possibly originated from bat-coronaviruses and was transmitted from animal-to-human, which later rapidly spread across the world through human movement. Globally, as of January 2021, there have been roughly 100 million confirmed cases of COVID-19, including around 2.2 million deaths. There is an urgent need for developing a vaccine for people throughout the world that fulfills all safety and efficacy requirements.

SARS-CoV-2 belongs to β coronavirus family which is found to have approximately 80% genetic sequence identical to SARS-CoV-1. The viral structure comprises spike(S) glycoprotein, envelope (E), and membrane (M) protein. Interestingly, SARS-CoV-2 is more infectious than SARS-CoV-1 as the virus has a higher reproductive number which leads to a rapid spread rate. The infection is caused by the virus binding to a target receptor in a host cell. Both SARS-CoV-1 and SARS-CoV-2 were reported to specifically interact with the angiotensin-converting enzyme 2 (ACE 2) receptor of host cells through the spike(S) protein. SARS-CoV-2 was reported to have a special structure in its surface proteins, thereby displaying a strong affinity to the ACE2 receptor. (Cevik M. et al., Bmj (2020), 371:m3862)

The currently available vaccine against COVID-19 is the mRNA vaccine synthesized from genetic material mimicking an actual part of the virus. Once the vaccine is administered to humans, it will trigger an immune response to produce antibodies against what is detected as a foreign substance. However, the expression levels of interest genes may vary among tissue types and there are challenges to clinically accomplish the required benefits. The available data collected from participants enrolled in an ongoing clinical trial showed that the vaccination generated side effects around the site of injection. The reactions could cause unfavorable pain and other symptoms, including redness, mild swelling and muscle pain, etc. Moreover, the vaccine needs to be stored at a temperature of minus 70° C., but many hospitals, especially in developing countries, do not have the necessary storage facilities. In addition, the cost of research and development for a novel vaccine against a new highly infectious disease like COVID-19 is extremely significant, which is difficult for developing countries to meet. Therefore, there is a need for an alternative production platform.

Recently, plants have been studied as a potential protein production platform. The fact is that plants offer several advantages over typical platforms, including rapid scalability, safety, and flexibility. Moreover, it provides an economical alternative to fermentation systems. Previous studies have reported the potential application of plant transient expression systems for producing a wide range of interest proteins with relatively high yield in a short period of time. This expression system is linearly and reliably scalable, simply by increasing the number of plants used in the experiment. (Stephenson M. J. et al., J Vis Exp (2018), (138): 58169)

The protein expressed through the plants can be applied for therapeutic substances, prophylactic medicine, vaccines or diagnostic reagents.

The production of receptor binding domain (RBD) of SARS-CoV-2 in Nicotiana benthamiana is disclosed herein. The RBD obtained from DNA encoding a viral fragment located in spike protein of SARS-CoV-2 is genetically modified with 8XHis tag at the C-terminus and then constructed into a germiniviral vector using specific restriction enzymes to create a recombinant vector. The recombinant vector was transformed into Agrobacterium tumefaciens, preferably strain GV3101, by electroporation. A recombinant Agrobacterium carrying the prepared vector was infiltrated into N. benthamiana plant leaves (Rattanapisit K. et al., Sci. Rep. (2010), 10:17698). However, such disclosure only revealed that the recombinant protein produced can specifically bind to the SARS-CoV-2 receptor, angiotensin-converting enzyme 2 (ACE 2), but there were no experiments showing immune response when said recombinant protein is administered to mammals. Additionally, such disclosure demonstrates an application of his-tag modified protein fragment to improve the protein productivity. Although, the application of tag modification of protein fragments can enhance protein expression and purification, some draw backs still remain. The protein fragment containing his-tag may not be suitable for administering to mammals, especially humans. The immune response will detect his-tag as foreign material as it is not naturally produced in humans, resulting in it being unsuitable for use as a human vaccine. Thus, the availability of a novel substance to trigger human antibodies against severe acute respiratory syndrome coronavirus 2 while not developing adverse effects is highly needed.

To improve the quality of protein expression and increase pharmacological characteristics, the ostepontin (OPN) protein contructed with Fc-based fusion protein to develop functional protein for tissue engineering application is disclosed. Said disclosure reveals that the fusion of Fc fusion domain in drug or therapeutic substance is found to have an advantageous effect in humans in terms of safety. Furthermore, the Fc fusion protein itself when fused with the protein of interest may increase plasma half-life so that the protein of interest may remain in the human body for a period of time. Besides, the fusion Fc improves the expression level and purified yield of the protein of interest. (Rattanapisit K. et al., Biotechnol Rep (2018), 21, e00312).

Concerning the safety and biological characteristics when administered to humans, the present invention also discloses tag modification. Instead of his-tag, Fc domain of immunoglobulin is introduced into the protein of interest. Fc domain of immunoglobulin is a known protein expressing a particular function and used to join end-to-end onto the protein of interest to facilitate protein localization and detection in the expression system. Fc domain demonstrates suitability for use in humans and it is widely used in Fc-based protein drugs and therapeutic monoclonal antibodies. Moreover, Fc domain can improve the yield and purity of recombinant protein produced in plants.

The present invention demonstrates a recombinant vector for producing immunogenic substance from plants which can induce an immune response in mammals against diseases caused by coronaviruses including COVID-19. Said recombinant vector comprises at least a fragment of SARS CoV-2 receptor binding domain protein (SARS CoV-2 RBD) and a fusion protein sequence. The recombinant vector is introduced into the plant cell by means of Agrobacterium sp., thereby the plant cell can express a recombinant protein which can act as an immunogenic substance. The recombinant protein of the present invention significantly demonstrates an ability to trigger immunogenicity in mammals which prevents infectious disease caused by severe acute respiratory syndrome coronavirus 2. Further, the method of inducing an immune response against SARS-CoV-2 in mammals is also provided herein. The present invention further demonstrates the use of such recombinant protein as a vaccine to prevent the coronavirus disease 2019 (COVID-19).

### SUMMARY OF THE INVENTION

According to one embodiment of the invention, the present invention provides a recombinant vector for producing immunogenic substance from plants comprising:
a) a severe acute respiratory syndrome coronavirus 2 surface glycoprotein sequence comprising amino acids 318-617 of SARS-CoV-2 receptor binding domain (SARS-CoV-2 RBD) protein sequence (SEQ ID NO: 2);
b) a fusion protein sequence which is fragment crystallizable (Fc) region from human immunoglobulin G1 (IgG1) (SEQ ID NO: 6); and
c) a plant expression vector.

In another exemplary embodiment, SARS-CoV-2 RBD protein sequence further comprises a signaling peptide (SEQ ID NO: 3) at N-terminus.

In another exemplary embodiment, SARS-CoV-2 RBD protein sequence further comprises a peptide linker at C-terminus for connecting SARS-CoV-2 RBD protein sequence to the fusion protein sequence (Fc).

In a preferred exemplary embodiment, the peptide linker comprises 1 to 5 tandem repeats of (GGGGS)ₙ(SEQ ID NO: 11).

In a preferred exemplary embodiment, the peptide linker is (GGGGS)₃ (SEQ ID NO: 5).

In another exemplary embodiment, the fusion protein sequence further comprises an endoplasmic reticulum retention motif at C-terminus.

In a preferred exemplary embodiment, the endoplasmic reticulum retention motif is a SEKDEL motif (SEQ ID NO:8).

In another exemplary embodiment, the plant expression vector derives from the bean yellow dwarf virus.

In another embodiment, host cell is transformed, infected, or induced with the recombinant vector, wherein the host cell is a bacterial cell.

In a preferred exemplary embodiment, the host cell is Agrobacterium tumefaciens.

In another embodiment, a plant cell infiltrated, transformed, infected, or induced with the hostcell.

In a preferred exemplary embodiment, the plant cell is Nicotiana sp.

In a preferred exemplary embodiment, the plant cell is Nicotiana benthamiana.

In another embodiment, a recombinant protein is produced from the plant cell.

In a preferred exemplary embodiment, the recombinant protein is severe acute respiratory syndrome-related coronavirus (SARSr-CoV) antigen.

In a preferred exemplary embodiment, the severe acute respiratory syndrome-related coronavirus (SARSr-CoV) antigen is severe acute respiratory syndrome coronavirus 2 (SARS-CoV-2) antigen.

In another exemplary embodiment, there is provided a use of the recombinant protein in the manufacture of a medicament for the prevention of the disease caused by severe acute respiratory syndrome-related coronavirus (SARSr-CoV).

In a preferred exemplary embodiment, there is provided a use of the recombinant protein in the manufacture of a medicament for the prevention of the disease caused by severe acute respiratory syndrome coronavirus 2 (SARS-CoV-2).

In another embodiment of the invention, a method of inducing a protective immune response against SARS-CoV-2 antigen comprising:
administering the recombinant protein according to the present invention to mammals in need thereof;
wherein the recombinant protein induces a protective immune response against challenge with SARS-CoV-2 in mammals.

In a preferred exemplary embodiment, the recombinant protein is prepared as an intramuscular injection.

In a preferred exemplary embodiment, the administering step comprises at least two vaccinations.

In a preferred exemplary embodiment, the administering step comprises two vaccinations.

In a preferred exemplary embodiment, the second vaccination is administered 14-28 days after the first vaccination.

In a preferred exemplary embodiment, the second vaccination is administered 21 days after the first vaccination.

### BRIEF DESCRIPTION OF THE DRAWING

FIG. 1 depicts the schematic representation of a recombinant vector, pBY-SARS-CoV-2 RBD-Fc, for expressing receptor binding domain of severe acute respiratory syndrome coronavirus 2 fused with a fusion protein sequence (SARS-CoV-2 RBD-Fc) protein.

FIGS. 2A, 2B, 2C and 2D depict the SDS-PAGE and Western blot analysis of the SARS-CoV-2 RBD-Fc recombinant protein expressed in Nicotiana benthamiana. The protein molecular weight marker (kDa) is indicated on the left. FIG. 2A depicts the SDS-PAGE analysis under reducing conditions of the total protein extracted from Nicotiana benthamiana infiltrated with pBY-SARS-CoV-2 RBD-Fc (lane 1) and the purified SARS-CoV-2 RBD-Fc recombinant protein (lane 2). FIG. 2B depicts the Western blot analysis of recombinant protein probed with a sheep anti-human gamma antibody conjugated with HRP under reducing conditions. The total protein extracted from Nicotiana benthamiana infiltrated with pBY-SARS-CoV-2 RBD-Fc is shown (lane 1), and the purified SARS-CoV-2 RBD-Fc recombinant protein is also shown (lane 2). FIG. 2C depicts the SDS-PAGE analysis under non-reducing conditions of the total protein extracted from Nicotiana benthamiana infiltrated with pBY-SARS-CoV-2 RBD-Fc (lane 1) and the purified SARS-CoV-2 RBD-Fc recombinant protein (lane 2). FIG. 2D depicts the western blot analysis of recombinant protein probed with a sheep anti-human gamma antibody conjugated with HRP under reducing conditions. The total protein extracted from Nicotiana benthamiana infiltrated with pBY01-SARS-CoV-2 RBD-Fc is shown (lane 1) and the purified SARS-CoV-2 RBD-Fc recombinant protein is shown (lane 2).

FIG. 3 depicts binding of SARS-CoV-2 RBD-Fc recombinant protein to angiotensin-convertinh enzyme 2 (ACE2) in vitro using the enzyme-linked immunosorbent assay (ELISA) technique.

FIG. 4 depicts the mouse vaccination schedule at day 0 and day 21. Antibodies in mouse sera were collected before vaccination, 14 days after the 1^{st} and 14 days after the 2^{nd} vaccination. Mouse splenocyte sample was collected 14 days after the 2^{nd} vaccination.

FIG. 5 depicts the antibody titer specific to SARS-CoV-2 RBD in sera of mice intramuscularly administered with SARS-CoV-2 RBD-Fc recombinant protein at each time point. FIG. 5A depicts mouse-IgG titer. FIG. 5B depicts mouse-IgG1 titer. FIG. 5C depicts mouse-IgG2a titer.

FIG. 6 depicts the neutralizing antibody titer against SARS-CoV-2 in sera of mice intramuscularly administered with SARS-CoV-2 RBD-Fc recombinant protein at each time point.

FIG. 7 depicts the T-cell lymphocyte responses against SARS-CoV-2 RBD in splenocytes of mice intramuscularly administered with SARS-CoV-2 RBD-Fc recombinant protein.

FIG. 8 depicts the monkey (Macaca fascicularis) vaccination schedule at day 0 and day 21. Antibodies in monkey sera were collected before vaccination, 14 days after the 1^{st} and 14 days after the 2^{nd} vaccination. Monkey splenocyte sample was collected 14 days after the 2^{nd} vaccination.

FIG. 9 depicts the IgG titer specific to SARS-CoV-2 RBD responses in sera of monkeys intramuscularly administered with SARS-CoV-2 RBD-Fc recombinant protein.

FIG. 10 depicts the neutralizing antibody titer against SARS-CoV-2 in sera of monkeys intramuscularly administered with SARS-CoV-2 RBD-Fc recombinant protein at each time point.

FIG. 11 depicts the T-cell lymphocyte responses against SARS-CoV-2 RBD in peripheral blood mononuclear cells of monkeys intramuscularly administered with SARS-CoV-2 RBD-Fc recombinant protein.

FIG. 12 depicts Coomassie staining of each group of excipients by SDS-PAGE, under reducing conditions, store for about 120 days under temperature about 2-8° C.

FIG. 13 depicts the 50% microneutralizing titer (MN₅₀) of SARS-CoV-2 RBD-Fc recombinant protein cynomolgus monkey immunized sera.

### DETAILED DESCRIPTION OF THE INVENTION

The present invention contains a Sequence Listing which is being submitted in computer readable form and which is hereby incorporated by reference in its entirety for all purposes. The txt file submitted herewith contains only one 30 KB file (sequence_18Mar2021.txt, created on 18 Mar. 2021))

The present invention provides a recombinant vector for producing an immunogenic substance which can induce an immune response in mammals against coronavirus disease 2019 (COVID-19). Said recombinant vector comprises at least a fragment of SARS CoV-2 receptor binding domain protein (SARS CoV-2 RBD) and a fusion protein sequence. The recombinant vector is introduced into the plant cell by means of Agrobacterium sp., thereby the plant cell can express a recombinant protein which can act as an immunogenic substance. The recombinant protein of the present invention significantly demonstrates an ability to trigger immunogenicity in mammals which prevents infectious disease caused by severe acute respiratory syndrome coronavirus 2. Further, the method of inducing an immune response against SARS-CoV-2 in mammals is also provided herein. The present invention further demonstrates the use of such recombinant protein as a vaccine to prevent the coronavirus disease 2019 (COVID-19). Still further provided are the pre-clinical results which show the efficiency of such recombinant protein when administering to mamals in vivo as a vaccine. The mammals exhibit a strong ability to induce, at least, immunoglobulin G (IgG) antibody, neutralizing antibody, and T-cell lymphocyte against SARS CoV-2 RBD. The present invention establishes promising results, thereby the development of a vaccine containing such recombinant protein for preventing the coronavirus disease 2019 (COVID-19) for mammals is disclosed. Details of the present invention can be elucidated according to the specification as follows.

Technical terms or scientific terms used herein have definitions as understood by those having an ordinary skill in the art, unless stated otherwise.

The disclosed SARS-CoV-2 RBD-Fc of the present invention includes conservative variants of the proteins and a person skilled in the art may recognize that some amino acids provided herein can be substituted by another amino acid without significantly changing the overall protein properties. A conservative substitution can be assessed by certain factors which include, without limition to, charge, hydrophobicity, hydrophilicity, size, covalent-bonding capacity, hydrogen-bonding capacity, or any combination thereof.

As used herein, the term “transformation”, “transformed”, “transforms”, “transforming”, “transfection”, “transfected”, “transfects”, “transfecting”, “transduction”, “transducted”, “transducts”, “transducting”, “insertion”, “inserted”, “inserts”, “inserting”, “transfer”, “transferred”, “transfers”, “transfering”, “infiltration”, “infiltrated”, “infiltrates”, and “infiltrating” refers to any process by which exogenous DNA enters, or is introduced or delivered into a host cell using methods well known in the art.

As used herein, the term “medicament” refers to any prophylactic, preventing, or protecting substances that, when administered to mammals, can stimulate the production of antibodies and provide immunity against disease caused by SARS-CoV-2. The medicament further ameliorates the effects of a future infection of SARS-CoV-2. The medicament can be obtained from the causative agent of SARS-CoV-2 disease, its products, or a synthetic substitute, and can be administered to mammals as an antigen without inducing the disease.

Equipment, apparatus, methods, or chemicals mentioned here means equipment, apparatus, processes, or chemicals commonly operated or used by those skilled in the art, unless explicitly stated otherwise that they are equipment, apparatus, methods, or chemicals specifically used in this invention. The use of singular or plural nouns with the term “comprising” in the claims or in the specification refers to “one” and also “one or more,” “at least one,” and “one or more than one.”

All compositions and/or processes disclosed and claimed are aimed to include aspects of the invention from actions, operation, modifications, or changing of any parameters without performing significantly different experiments from this invention, and obtaining similar objects with the same utilities and results of the present invention according to persons skilled in the art, although without mention of the claims specifically. Therefore, substitution or similar objects to the present invention including minor modifications or changes which can be clearly seen by persons skilled in the art should be considered within the scope, spirit, and concept of the invention as appended claims.

Throughout this application, the term “about” is used to indicate that any value presented herein may potentially vary or deviate. Such variation or deviation may result from errors of apparatus, methods used in calculation, or from an individual operator implementing apparatus or methods. These include variations or deviations caused by changes of the physical properties.

The following is a detailed description of the invention without any intention to limit the scope of the invention.

According to one embodiment of the invention, the present invention provides a recombinant vector for producing immunogenic substance from plants comprising:
a) a severe acute respiratory syndrome coronavirus 2 surface glycoprotein sequence comprising amino acids 318-617 of SARS-CoV-2 receptor binding domain (SARS-CoV-2 RBD) protein sequence (SEQ ID NO: 2);
b) a fusion protein sequence which is fragment crystallizable (Fc) region from human immunoglobulin G1 (IgG1) (SEQ ID NO: 6); and
c) a plant expression vector.

The spike protein of SARS-CoV-2 consists of an S1 subunit and S2 subunit in each spike, where S1 subunit comprises receptor-binding domain (RBD) which plays crucial roles in viral infection (Lan J. et al. Nature (2020), 518:215-220). Once the virus enters the host, the viral RBD specifically binds to ACE2 receptor, which is located on the epithelial cells of the nasal cavity of the host, before passing through the respiratory tract to reach the lungs (Shah V. K. et al., Front Immunol (2020), 11:1949).

The transmembrane glycosylated protein of spike composed of 1273 amino acids where the receptor binding domain (RBD) of SARS-CoV-2 is from residues 319-591. SARS-CoV-2 RBD interacts with ACE2 which acts as a SARS-CoV-2 receptor for mediating the invasion of virus to host cell (Auge C. R. et al., Sci Rep (2020), 10, 21779). In another exemplary embodiment, SARS-CoV-2 RBD protein sequence further comprises a signaling peptide (SEQ ID NO: 3) at N-terminus.

In another exemplary embodiment, SARS-CoV-2 RBD protein sequence further comprises a peptide linker at C-terminus for connecting SARS-CoV-2 RBD protein sequence to the fusion protein sequence (Fc).

One advantage of fusing the fusion protein sequence at C-terminus of SARS-CoV-2 RBD-Fc protein sequence is to allow a high level of the protein expression.

Suitable fusion protein sequence plays a role in the protein expression as it provides rapid and simple detection of protein expression. Several studies indicate that the fusion protein sequence based on Fc fragment from human immunoglobulin G1 (IgG 1) in the recombinant protein can boost the binding affinity and provide stability of protein expression.

The fusion protein sequence as disclosed in this invention comprises amino acids represented in SEQ ID NO: 6.

Preferably, the N-terminus of the fusion protein sequence is cleaved with BamHI restriction enzyme and fused onto the C-terminus of SARS-CoV-2 RBD. Preferably, the C-terminus of the fusion protein sequence is cleaved with SacI restriction enzyme.

The present invention further discloses SARS-CoV-2 RBD protein sequence comprising a peptide linker at C-terminus for connecting SARS-CoV-2 RBD protein sequence to the fusion protein sequence. SARS-CoV-2 RBD and the fusion protein sequences are linked through a peptide linker comprising (GGGGS)ₙ(SEQ ID NO: 11).

In another exemplary embodiment, the peptide linker comprises 1 to 5 tandem repeats of (GGGGS)ₙ(SEQ ID NO: 11).

In a preferred exemplary embodiment, the peptide linker is (GGGGS)₃.

The peptide linker as disclosed in this invention comprises amino acids represented in SEQ ID NO: 5

The SARS-CoV-2 RBD-peptide linker-Fc as disclosed in this invention comprises amino acids represented in SEQ ID NO: 7

In another exemplary embodiment, the fusion protein sequence further comprises an endoplasmic reticulum retention motif at C-terminus.

The present invention also discloses a plant expression system to express the protein of interest, by modifying SARS-CoV-2 RBD-Fc protein sequence with the particular motif as described below to increase the level of protein expression.

The endoplasmic reticulum (ER) lumen of plants, where molecular chaperones and enzymes reside therein, plays a significant role in supporting proper protein folding which makes proteins function appropriately. Additionally, in order to improve SARS-Cov-2 RBD-Fc expression, it is advantageous to target proteins through the secretory pathway. Therefore, the ER-targeting is advantageous for the production of SARS-CoV-2 RBD-Fc at high yield. (Hamorsky K. T. et al. 2015. Sci. Rep. 5)

In a preferred exemplary embodiment, the endoplasmic reticulum retention motif is a SEKDEL motif (SEQ ID NO: 8).

The endoplasmic reticulum retention motif is linked to the C-terminus of Fc region and another end on such motif is linked to the plant expression vector via polymerase chain reaction (PCR).

The SARS-CoV-2 RBD-Fc comprising a peptide linker and KDEL motif is represented in SEQ ID NO: 10

The plant expression vector is generally constructed with an origin of replication (ori), antibiotic resistant genes, restriction endonuclease sites, a promoter, and transmissability, etc., and can be exploited in various protein expression applications. In particular, the plant expression vector plays a role as a carrier to introduce genes or nucleic acid sequences into plant cells. Such vectors can transiently produce a high yield of protein of interest, allow rapid production and provide a simply purified process. Accordingly, the present invention also discloses a plant expression vector comprising specific nucleic acid which provides particular characteristics and makes said vector suitable for introducing one or more genetic materials into the host cell.

In particular, the present invention provides the plant expression vector comprising the right and left borders of the T-DNA region transferred by Agrobacterium, RB and LB; P35S: Cauliflower Mosaic Virus (CaMV) 35S promoter, NbPsalK2T1-63 5′UTR: 5′ untranslated region, RBD: SARS-CoV-2 RBD, Tag: Fc region, Ext3′FL: 3′ region of tobacco extension gene, Rb7 5′ del: tobacco RB7 promoter, SIR: short intergenic region of BeYDV, LIR: long intergenic region of BeYDV, C2/C1: Bean Yellow Dwarf Virus (BeYDV) ORFs C1 and C2 encoding for replication initiation protein (Rep) and RepA, TMVΩ 5′-UTR: 5′ untranslated region of tobacco mosaic virus Ω, P19: the RNA silencing suppressor from tomato bushy stunt virus; PinII 3′: the terminator from potato proteinase inhibitor II gene.

The SARS-CoV-2 RBD-Fc comprising a peptide linker and SEKDEL motif described above is inserted into the plant expression vector to create a recombinant vector, pBY-SARS-CoV-2 RBD-Fc, using XbaI and SacI restriction enzymes. The recombinant vector is amplified by introducing into competent E. coli cells according to a standard protocol generally known in the art including, without limition to, transfection, insertion, transformation, and transduction. The method for introducing the recombinant vector into competent E. coli cells as disclosed herein and can be found in, for example, Rattanapisit K. et al., Sci. Rep. (2010), 10:17698. After that, the recombinant vector, pBY-SARS-CoV-2 RBD-Fc, is introduced into Agrobacterium sp., where the inserted SARS-CoV-2 RBD-Fc is intregated into Agrobacterium genome.

It will be understood by those skilled in the art that any transformation method may be utilized within the definitions and scope of the invention. Such methods can include heat shock and electroporation.

In another exemplary embodiment, the host cell is a bacterial cell.

In a preferred exemplary embodiment, the host cell is Agrobacterium tumefaciens, preferably strain GV3101.

The recombinant Agrobacterium strain GV3101 is infiltrated into tobacco leaves. The standard protocol for infiltration is generally know in the art and can be found in, for example, Rattanapisit K. et al., Sci. Rep. (2010), 10:17698.

In another exemplary embodiment, the plant cell is Nicotiana sp.

In a preferred exemplary embodiment, the plant cell is Nicotiana benthamiana.

After 4 days, the infiltrated tobacco leaf can be harvested and processed to extract and purify the recombinant protein.

In another exemplary embodiment, the recombinant protein is severe acute respiratory syndrome-related coronavirus (SARSr-CoV) antigen.

In a preferred exemplary embodiment, the severe acute respiratory syndrome-related coronavirus (SARSr-CoV) antigen is severe acute respiratory syndrome coronavirus 2 (SARS-CoV-2) antigen.

In another exemplary embodiment, there is provided a use of the recombinant protein in the manufacture of a medicament for the prevention of the disease caused by severe acute respiratory syndrome-related coronavirus (SARSr-CoV).

In a preferred exemplary embodiment, there is provided the use of the recombinant protein in the manufacture of a medicament for the prevention of the disease caused by severe acute respiratory syndrome coronavirus 2 (SARS-CoV-2).

Also disclosed herein are methods for inducing an immune response to SARS-CoV-2 using SARS-CoV-2 RBD-Fc in the present invention as an antigen. Said SARS-CoV-2 RBD-Fc can be further contained in vaccine composition to prevent the disease caused by coronavirus, which is coronavirus disease 2019 (COVID-19). Further, various formulations of the recombinant protein, suitable excipients, stabilizers, and the like that may be added are known by persons of ordinary skill in the art.

In one embodiment, a method of inducing a protective immune response against SARS-CoV-2 antigen comprising:
administering the recombinant protein according to the present invention to mammals in need thereof:
wherein the recombinant protein induces a protective immune response against challenge with SARS-CoV-2 in mammals.

In a preferred exemplary embodiment, the recombinant protein is prepared as an intramuscular injection.

In a preferred exemplary embodiment, the administering step comprises at least two vaccinations.

In a preferred exemplary embodiment, the administering step comprises two vaccinations.

In a preferred exemplary embodiment, the second vaccination is administered 14-28 days after the first vaccination.

In a preferred exemplary embodiment, the second vaccination is administered 21 days after the first vaccination.

The method of inducing a protective immune response in mammals is further detailed by means of the following examples.

Hereafter, examples of the invention are shown without any purpose to limit any scope of the invention.

### EXAMPLE 1

### Construction and Cloning of pBY-SARS-CoV-2 RBD-Fc Recombinant Vector

First, amino acid sequences encoding receptor binding domain of severe acute respiratory syndrome virus 2 (SARS-CoV-2 RBD) were codon-optimized to improve expression efficiency in Niocotiana benthamiana.

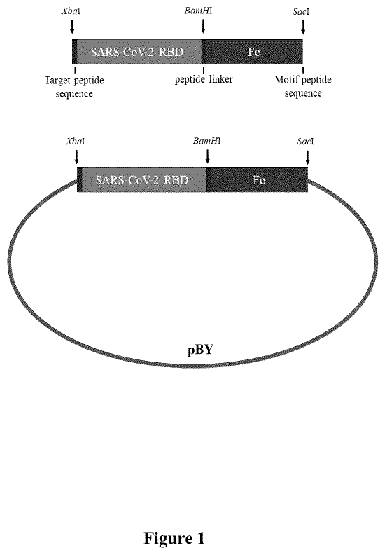

To construct SARS-CoV-2 RBD-Fc protein, the sequence comprising amino acids 318-617 (SEQ ID NO: 2) of surface glycoprotein of SARS-CoV-2 (SEQ ID NO: 1) was designed for receptor binding domain (RBD). Then, the codon-optimized RBD of SARS-CoV-2 (encoded to amino acid in SEQ ID NO: 4) containing signal peptide at N-terminus (SEQ ID NO: 3) with XbaI restriction enzyme site and GGGS at C-terminus with BamHI restriction enzyme site was synthesized (manufactured by Genewiz, Inc, China). The Fc fragment from human immunoglobulin G1 (Fc region) (encoded to amino acid in SEQ ID NO: 6) was designed to contain GGGS×2 at N-terminus with BamHI restriction enzyme site and SEKDEL motif (SEQ ID NO: 8) at C-terminus with SacI restriction enzyme site (encoded to amino acid in SEQ ID NO: 9). Both SARS-CoV-2 RBD and Fc region were ligated via BamHI site. Then, SARS-CoV-2 RBD-Fc (encoded to amino acid in SEQ ID NO: 10) was ligated via XbaI and SacI sites to pBY plant expression vector to generate a recombinant vector, pBY-SARS-CoV-2 RBD-Fc, as shown in FIG. 1.

Subsequently, the recombinant vector was introduced into Escherichia coli strain DH 108 competent cells by heat shock transformation. The transformed E. coli was plated on Luria Bertani (LB) agar containing kanamycin and incubated at about 37° C. for about 24 hours. Several colonies were picked and verified by PCR using primers specific to SARS-CoV-2 RBD-Fc gene, BsaI-W-F and SacI-KD-R. The selected colonies were cultured in LB broth containing kanamycin and incubated at about 37° C. for about 16 hours while shaking at about 200 rpm. The extraction of the recombinant vector was performed according to DNA-spin™ Plasmid DNA Purification Kit (iNtron Biotechnology, South Korea).

The recombinant vector, pBY-SARS-CoV-2 RBD-Fc, was introduced into Agrobacterium tumefaciens strain GV3101 by electroporation. The voltage was set at about 2 kilovolts for about 2 minutes. The transformed A. tumefaciens was plated on LB agar containing mixed rifampicin, gentamycin, and kanamycin antibiotics, then incubated at about 28° C. for approximately 48 hours. Several colonies were verified by PCR using primers specific to the SARS-CoV-2 RBD-Fc gene. The selected colonies were cultured in LB broth containing mixed antibiotics as stated above and incubated at about 28° C. for about 24 hours while shaking at approximately 200 rpm. A recombinant A. tumefaciens was prepared for expression in plants.

### EXAMPLE 2

### Transformation of Recombinant A. Tumefaciens into by Agrobacterium-Mediated Transformation

The recombinant A. tumefaciens carried pBY-SARS-CoV-2 RBD-Fc vector was cultured in 4 L of LB broth containing mixed rifampicin, gentamycin, and kanamycin antibiotics and incubated at about 28° C. for about 24 hours while shaking at about 200 rpm. Subsequently, the recombinant A. tumefaciens was dissolved in infiltration buffer (1×Infiltration buffer: 10 mM of 2-(N-morpholino), ethanesulfonic acid (MES), 10 mM of MgSO₄, at pH 5.5) until the prepared mixture reached an optical density at 600 nm (A₆₀₀) of 0.1. The agroinfiltration technique was performed under vacuum infiltration to infiltrate the recombinant A. tumefaciens into 4-6 week-old tobacco leaves (Nicotiana benthamiana). The seed was obtained from Biodesign Institute at Arizona State University, School of Life Sciences, Faculty of Biomedicine & Biotechnology, Tempe, AZ, USA. The tobacco leaves were incubated at about 28° C. under the light for about 16 hours a day for about 4 days in a growth chamber and were harvested for determination of protein expression and purification.

### EXAMPLE 3

### SARS-CoV-2 RBD-Fc Recombinant Protein Extraction

The infiltrated leaves were extracted with phosphate buffered saline (1× PBS) (about 137 mM NaCl, about 2.68 mM KCl, about 10.1 mM of Na₂HPO₄, about 1.76 mM KH₂PO₄ at pH 7.4) to obtain SARS-CoV-2 RBD-Fc recombinant protein. The ratio of the infiltrated leaf weight to the extraction buffer is about 1:2. Crude extract was separated, and supernatant was centrifuged at about 10,000 rpm, about 4° C. for about 1 hour. Then the obtained supernatant was filtered with 0.45 micron S-Pak membrane (Merck, Massachusetts, USA) and the filtrate was collected for protein purification.

### EXAMPLE 4

### SARS-CoV-2 RBD-Fc Recombinant Protein Purification Using Affinity Chromatography

To purify SARS-CoV-2 RBD-Fc recombinant protein, the column for purifying protein was prepared by adding rProtein A Sepharose Fast Flow purification resin™ (cytiva, MA, USA) into the column, then washed and adjusted the conditions of the mixture contained in the column with deionized water and 1× PBS, pH 7. 4 respectively. The filtrate from protein extraction was poured into the prepared column where SARS-CoV-2 RBD-Fc recombinant protein is bound with the rProtein A Sepharose Fast Flow purification resin™. The column was generously washed with 1× PBS, pH 7.4. The SARS-CoV-2 RBD-Fc recombinant protein was eluted from rProtein A Sepharose Fast Flow purification resin™ with about 5 ml of about 0.1 M glycine buffer, pH 3, about 1 ml each for about 5 times and neutralized with about 1.5 M Tris-HCl, pH 8.8. The protein concentration was enhanced by adding Amicon® ultracentrifugal filter (Merck, USA). The purified SARS-CoV-2 RBD-Fc recombinant protein was filtered with 0.22 micron membrane (Millipore, USA) to remove contaminant.

### EXAMPLE 5

### SDS-PAGE and Western Blot

Sodium dodecyl sulfate-polyacrylamide gel electrophoresis (SDS-PAGE) was performed on 10% acrylamide gels. The purified SARS-CoV-2 RBD-Fc protein was mixed with Z buffer (Z buffer: about 1M Tris-HCl pH 6. 8, about 12% Sodium Dodecyl Sulfate, about 10% glycerol, Bromophenol blue) and incubated a mixture about 95° C. for about 5 minutes. Gels were run for about 1.5 hours at about 100 V in buffer (1× running buffer: about 0.025M Tris base, about 0.19 M glycine, and about 0.003 M SDS). The SARS-CoV-2 RBD-Fc recombinant protein was visualized by Coomassie's brilliant blue staining and Western blotting was performed to determine the SARS-CoV-2 RBD-Fc recombinant protein.

The SARS-CoV-2 RBD-Fc recombinant protein on the gel was transferred onto a nitrocellulose membrane (Biorad, USA) facilitated by about 100 V electric current for about 2 hours in 1× Transfer buffer (about 0. 01M Tris base, about 0.01M glycine, methanol). The membrane was fully blocked for about 45 minutes at room temperature with about 5% skim milk dissolved in 1× PBS and incubated for about 2 hours at room temperature with the sheep anti-human Gamma chain-HRP conjugate antibody (The Binding Sites, UK) diluted (at about 1:5000) in about 3% skim milk. The membrane was washed about 3 times with 1× PBS, and about 0.05% Tween 20. The membranes were developed using Enhanced Chemiluminescence (ECL reagents: Abcam, UK). Medical X-ray Green/MXG film (Carestream Health, China) as chemiluminescence was used for detecting SARS-CoV-2 RBD-Fc protein.

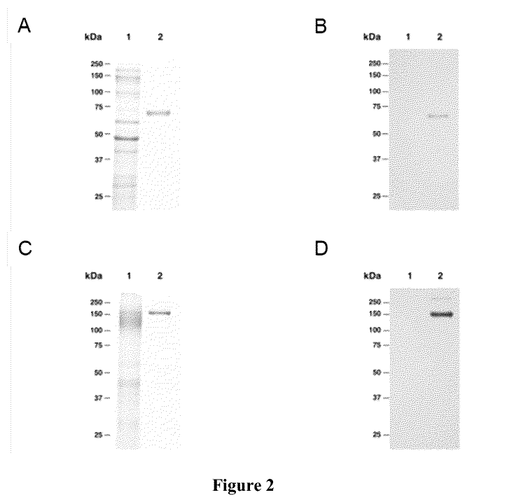

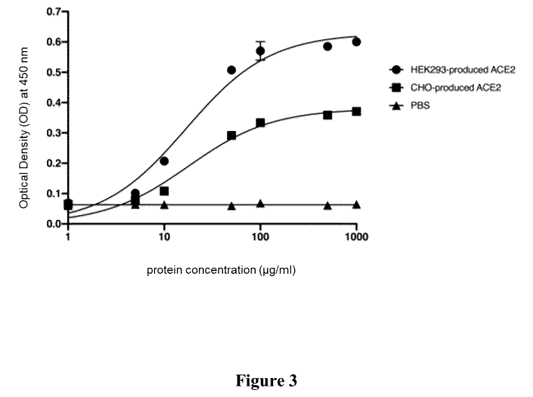

The expression of SARS-CoV-2 RBD-Fc recombinant protein in tobacco leaves (Nicotiana benthamiana) was shown in FIG. 2. The SDS-PAGE analysis showed the total protein extracted from Nicotiana benthamiana infiltrated with pBY-SARS-CoV-2 RBD-Fc (lane 1) and the purified SARS-CoV-2 RBD-Fc (lane 2) under reducing conditions (FIG. 2A) showed the size of about 75 kDa. Meanwhile, the purified SARS-CoV-2 RBD-Fc recombinant protein band (lane 2) under non-reducing conditions (FIG. 2C) showed the size of about 150 kDa. The results suggested that SARS-CoV-2 RBD-Fc recombinant protein were formed as protein dimer which bound together through disulfide bonds. Western blot analysis of SARS-CoV-2 RBD-Fc protein probed with a sheep anti-human gamma antibody conjugated with HRP showed the corresponding results such that the SARS-CoV-2 RBD-Fc protein comprised the size of about 75 kDa under reducing conditions, and about 150 kDa under non-reducing conditions. These suggested the expression level of SARS-CoV-2 RBD-Fc recombinant protein was about 25 μg per gram of fresh leaf weight. The results also suggested the SARS-CoV-2 RBD-Fc recombinant protein could specifically bind to tested antibodies.

### EXAMPLE 6

### Indirect Enzyme-Linked Immunosorbent Assay (Indirect ELISA)

The SARS-CoV-2 RBD-Fc recombinant protein concentration was determined using indirect ELISA assay. The 96-well plate (Greiner Bio-One GmbH) was pre-coated with commercially available standard HEK293-produced SARS-CoV-2 RBD-Fc (R&D Systems, USA) and about 50 μl of SARS-CoV-2 RBD-Fc recombinant protein under a temperature of about 4° C. The 96-well plate was washed about 3 times with about 200 μl 1× PBS-T. Then, about 5% w/v, 200 μl, at about 37° C. of skim milk solution was added into the 96-well plate and left for about 2 hours and the plate was washed about 3 times with about 200 μl 1× PBS-T. SARS CoV-2 Spike Protein (RBD) Chimeric Recombinant Rabbit Monoclonal antibody (ThermoFisher scientific, USA) was diluted (at about 1:2000) in 1× PBS and the mixture was added into the 96-well plate under a temperature of about 37° C., then left for approximately 2 hours. Subsequently, goat anti-rabbit-HRP fusion (BosterBio, USA) was diluted (1:2000) in 1× PBS and the mixture was added onto the 96-well plate under a temperature of about 37° C., then left for about 1 hour. The 96-well plate was washed about 3 times with 1× PBS-T and 3,3′, 5,5′-Tetramethylbenzidine (TMB) solution (Promega, USA) was added, in which the mixture began to transform into blue color. About 1 M H₂SO₄ was dropped onto the 96-well plate to stop a reaction. The 96-well plate was then placed in a 96-well microplate reader (Molecular Devices, USA) at about 450 nm absorbance to determine SARS-CoV-2 RBD-Fc recombinant protein concentration.

The binding affinity of the SARS-CoV-2 RBD-Fc protein to angiotensin converting enzyme 2 (ACE2) was analyzed. The 96-well plate was pre-coated with about 2 μg/ml, about 50 μl of each commercially available HEK293-ACE2 (Abcam, UK) and CHO-ACE2 (InvivoGen, California, USA) at about 4° C. After incubation, the coating buffer was discarded and the plate was blocked with about 5% skim milk in 1× PBS for about 2 hours under a temperature of about 37° C. The plate was then incubated with SARS-CoV-2 RBD-Fc recombinant protein at the concentration of about 1,000, 500, 100, 50, 10, 5 and 1 μg/ml. for about 2 hours under a temperature of about 37° C. Next, an antibody specific to SARS-CoV-2 RBD-Fc protein mixed with 1× PBS at the ratio of about 1:1000 was added and incubated for about 2 hours under a temperature of about 37° C. An anti-human Kappa chain-HRP fusion (SouthernBiotech, USA) diluted (at about 1:1000) in 1× PBS was added to the plate which was then incubated for 1 hour under a temperature of about 37° C. Finally the plate was washed with 1× PBST. The reaction was developed with TMB solution (Promega, USA) and stopped by about 1 M of H₂SO₄. The absorbance at about 450 nm (A450) was measured by the 96-well microplate reader (Molecular Devices, USA).

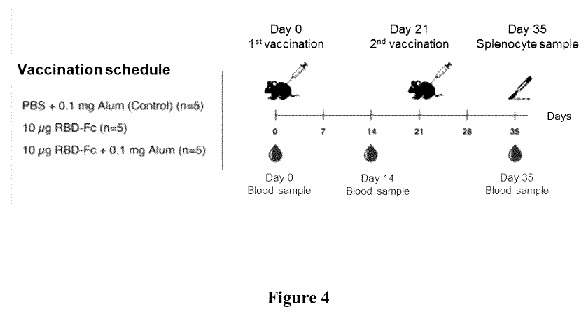

FIG. 4 showed that SARS-CoV-2 RBD-Fc recombinant protein can bind to the receptor of SARS-CoV-2 (ACE2) produced from HEK-293 cells and CHO cells. The result indicated the authenticity and proper folding of the SARS-CoV-2 RBD-Fc recombinant protein. Interestingly, the SARS-CoV-2 RBD-Fc recombinant protein in the present invention possessed biological properties similar to intrinsic SARS-CoV-2 RBD in coronavirus.

### EXAMPLE 7

### Mouse Immunization and Sample Collection

Mouse immunization protocol was investigated and certified by the Institutional Animal Care and Use Committee (IACUC), Faculty of Medicine, Chulalongkorn University, Thailand. 7 week-old female ICR mice (N=15) were divided into 3 groups. Mice were administered intramuscularly (IM) at the anterior tibialis site with either 1× PBS formulated with about 0.1 mg alum adjuvant (control, N=5), about 10 μg/mouse of SARS-CoV-2 RBD-Fc recombinant protein (N=5), or about 10 μg/mouse of SARS-CoV-2 RBD-Fc recombinant protein formulated with about 0.1 mg alum adjuvant (InvivoGen, USA) (N=5). Each group was subjected to the 2^{nd} vaccination with about 10 μg/mouse of the same formulation at about 21-day-intervals. To detect SARS-CoV-2 RBD-Fc specific antibodies and neutralizing antibodies, mouse sera was collected before vaccination, about 14 days after 1^{st} and about 14 days after 2^{nd} vaccination. Mouse splenocyte sample was only collected about 14 days after 2^{nd} vaccination.

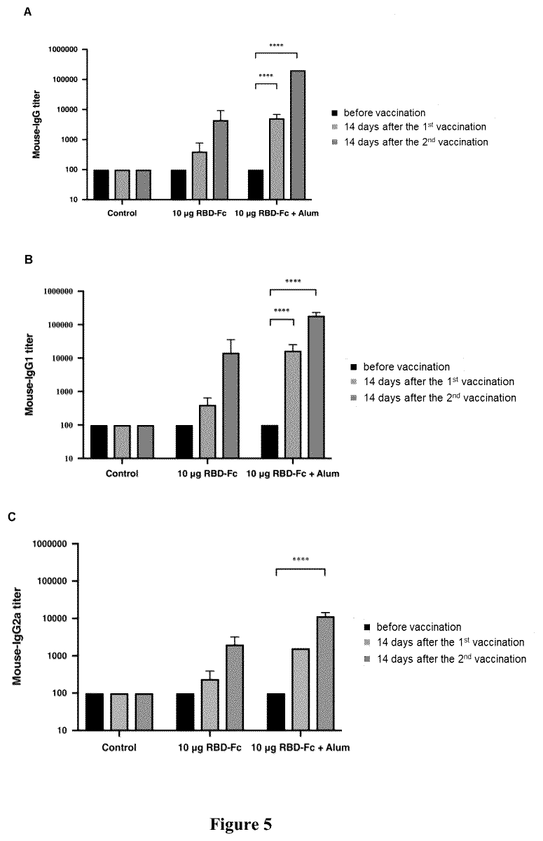

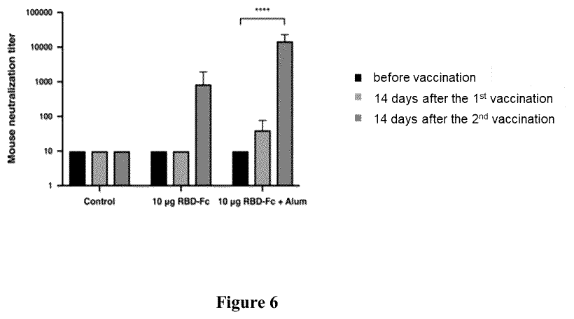

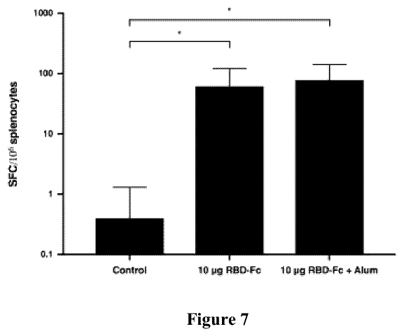

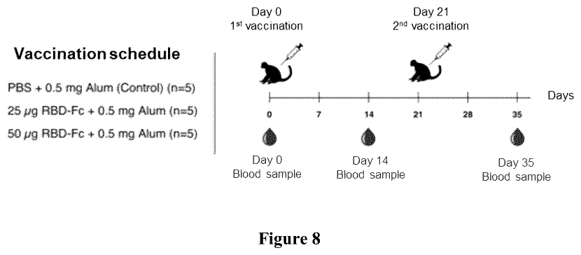

FIG. 5 showed mouse-IgG titers (FIG. 5A), mouse-IgG1 titers (FIG. 5B) and mouse-IgG2a titers (FIG. 5C) specific to SARS-CoV-2 RBD protein which were induced after about 14 days from the 1^{st} vaccination with about 10 μg/mouse of SARS-CoV-2 RBD-Fc recombinant protein. The efficiency of immunization was even better in mice administered with 10 μg/mouse of SARS-CoV-2 RBD-Fc recombinant protein formulated with about 0.1 mg alum adjuvant. The results showed that antibody titers specific to SARS-CoV-2 RBD were produced after 14 days from the 2^{nd} vaccination at a significant level compared to the control group. These results confirmed that SARS-CoV-2 RBD-Fc recombinant protein in the present invention can be used for efficiently triggering immunogenicity after vaccinating twice.

### EXAMPLE 8

### Monkey (Macaca fascicularis) Vaccination and Sample Collection

Monkey (Macaca fascicularis) vaccination protocol was investigated and certified by National Primate Research Center of Thailand-Chulalongkorn University NPRCT-CU, accreditted by AAALAC International. 2.5-3.5 year-old monkeys (N=13) weighing 2.18-3.17 kg, were divided into 3 groups. Monkeys were administered intramuscularly (IM) with either 1× PBS formulated with about 0.5 mg alum adjuvant (control, N=3), about 25 μg/monkey of SARS-CoV-2 RBD-Fc recombinant protein formulated with about 0.5 mg alum (N=5), or about 50 μg/mouse of SARS-CoV-2 RBD-Fc recombinant protein formulated with about 0.5 mg alum adjuvant (InvivoGen, USA) (N=5). Each group was subjected to the 2^{nd} vaccination with the same formulation at about a 21-day-interval. To detect SARS-CoV-2 RBD-Fc-specific antibodies and neutralizing antibodies, monkey sera was collected before vaccination, 14 days after the 1^{st} and 14 days after the 2^{nd} vaccination.

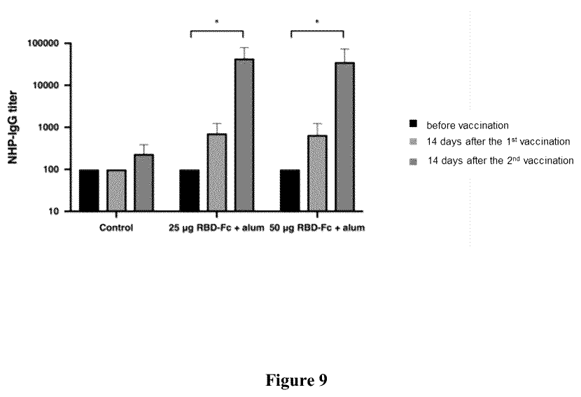

FIG. 9 showed monkey-IgG titers specific to SARS-CoV-2 RBD which were induced about 14 days after the 1^{st} vaccination. The results also showed that monkey-IgG titers specific to SARS-CoV-2 RBD were produced about 14 days after the 2^{nd} vaccination at a significant level. There was no relation between doses of SARS-CoV-2 RBD-Fc recombinant protein on triggering immunogenicity in monkeys both at about 14 days after the 1^{st} and about 14 days after the 2^{nd} vaccination. The lowest dose of SARS-CoV-2 RBD-Fc recombinant protein to trigger immunogenicity in monkeys was about 25 μg.

### EXAMPLE 9

### Detection of Antibodies Specific to SARS-CoV-2 RBD-Fc Responses in Collected Animal Sera Sample After Immunization by ELISA

ELISA was performed to analyze SARS-CoV-2 RBD-Fc antibody responses from collected animal sera. The 96-well plate was pre-coated with about 2 μg/ml, about 50 μl of SARS-CoV-2 spike protein (RBD) from Sf9 cells (GenScript, USA) about 4° C. overnight. After incubation, the coating buffer was discarded, washed about 3 times with about 200 μl 1× PBS-T, and the plate was blocked with about 200 μl of 5% skim milk in 1× PBS for about 2 hours at about 37° C. The plate was then incubated with collected animal sera (i.e. sera collected either from administered mice or administered monkeys), serially two-fold diluted in 1× PBS until it reached the endpoint titer, for about 2 hours under a temperature of about 37° C. Next, 3 types of antibodies specific to collected mouse sera, which were goat anti-mouse IgG HRP conjugate antibody (Jackson ImmunoResearch, USA), goat anti-mouse IgG1 (HRP) antibody, and goat anti-mouse IgG2a heavy chain (HRP) antibody (Abcam, UK) diluted (at about 1:2,000) in 1× PBS were added into the plate and incubated for about 1 hour under a temperature of about 37° C. Meanwhile in collected monkey sera, only goat anti-monkey IgG HRP conjugation antibody (Abcam, UK) diluted (at about 1:2,000) in 1× PBS was added into the plate and incubated for 1 hour under a temperature of about 37° C. The reaction was developed with TMB solution (Promega, USA) and stopped by about 1M H₂SO₄. The absorbance at about 450 nm (A450) was measured by the 96-well microplate reader (Molecular Devices, USA).

### EXAMPLE 10

### Detection of Neutralizing Antibody Response in Collected Animal Sera Sample After Immunization by Microneutralization Assay

All the experiments with live SARS-CoV-2 virus were performed at a certified biosafety level 3 facility, Department of Microbiology, Faculty of Science, Mahidol University, Thailand. The experimental protocol was approved by Mahidol University and all methods were performed in accordance with the relevant guidelines and regulations.

Neutralizing antibody titers of animal sera against SARS-CoV-2 RBD-Fc recombinant protein were detected. First, Vero E6 cells were prepared in DMEM (Dulbecco's Modified Eagle's medium: about 10% heat-inactivated FBS, about 100 U/mL of penicillin and about 0.1 mg/mL of streptomycin) under a temperature of about 37° C. and about 5% CO₂ in a humidified incubator, incubated in 96-well plate with about 1×10⁴ cells, and washed.

A positive convalescent serum of a COVID-19 patient was approved for use as a clinical specimen by the Faculty of Medicine Ramathibodi Hospital, Mahidol University. Informed consent was waived by the Institutional Review Board that approved the present study. The collected animal sera samples and the positive serum were first serially diluted (at about 1:10) in DMEM and then serially two-fold diluted to achieve varying concentrations, followed by individual incubation with 100 TCID₅₀ in DMEM of the SARS-CoV-2 virus for about 1 hour under a temperature of about 37° C.

The serum of the COVID-19 patient and the collected animal sera sample virus incubated with SARS-CoV-2 virus were added to a prepared 96-well plate containing about 1×10⁴ Vero E6 cells/well and cultured under a temperature of about 37° C., about 5% CO₂, for about 2 days. The plates were washed three times with 1× PBS, then incubated with ice-cold of about 1:1 methanol/acetone fixative for about 20 minutes under a temperature of about 4° C. then washed about 3 times with 1× PBST. Blocking reagent (2% bovine serum albumin, BSA) was added to the wells, and plates were incubated for about 1 hour at 25-30° C. After washing, antibodies specific to SARS-CoV/SARS-CoV-2 nucleocapsid (N) monoclonal antibody (SinoBiological, USA) diluted (at about 1:5,000) in 1× PBS were added and the samples were incubated under a temperature of about 37° C. for about 1 hour. Then, HRP-conjugated goat anti-rabbit polyclonal antibody (Dako, Denmark) diluted (1:2,000) in 1× PBS was added. After incubation under a temperature of about 37° C. for about 1 hour, a reaction was developed with KPL Sureblue™ TMB substrate (SeraCare, USA) and stopped by about 1 N of HCl. The absorbance at about 450 nm (A₄₅₀) and about 620 nm (A₆₂₀) were measured by the Sunrise™ microplate reader (Tecan, Switzerland). Neutralization titers, determined from the last diluted titer above A cut point, were calculated using the following equation:

$${A}_{cut point} = \frac{{A}_{virus control} - {A}_{cell control}}{2} + {A}_{cell control}$$

where: A_{cut point}=The difference in absorbance between A₄₅₀ and A₆₂₀ nm used for determining a positive control.
A_{virus control}=The difference in absorbance between A₄₅₀ and A₆₂₀ in a positive control of virus.
A_{cell control}=The difference in absorbance between A₄₅₀ and A₆₂₀ in negative control of cell culture.

An immunofluorescence assay result indicated that the neutralizing activity of administered mouse sera against SARS-CoV-2 RBD was shown about 14 days after the 1^{st} vaccination, and even more efficiently neutralized SARS-CoV-2 RBD about 14 days after the 2^{nd} vaccination. The efficiency of neutralizing activity was significant in mice administered with about 10 μg/mouse of SARS-CoV-2 RBD-Fc recombinant protein formulated with about 0.1 mg alum adjuvant compared to the control group. These results confirmed that SARS-CoV-2 RBD-Fc recombinant protein of the present invention exhibited an ability for efficiently triggering neutralizing activity after vaccinating twice.

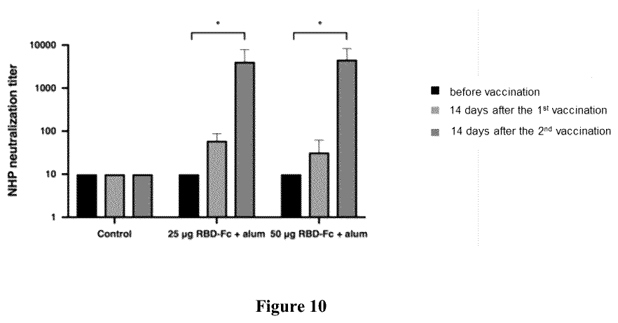

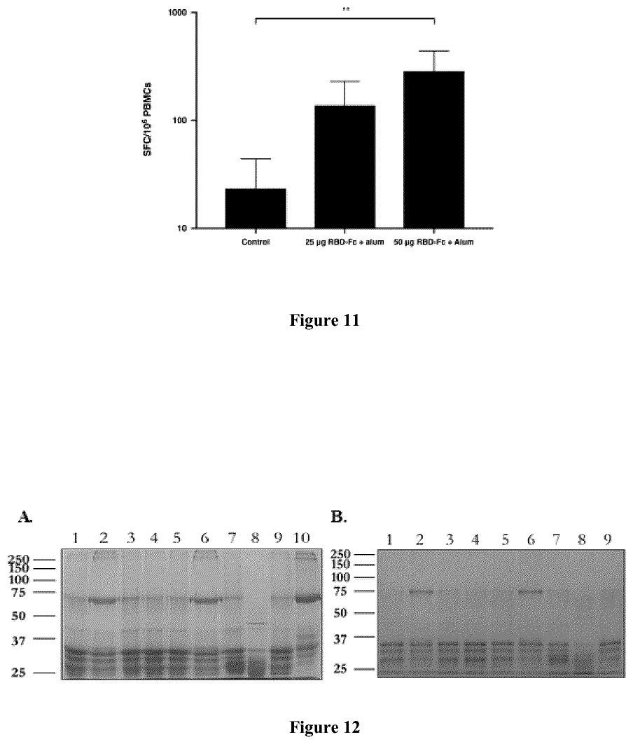

Further, FIG. 10 indicated the neutralizing activity of administered monkey sera against SARS-CoV-2 RBD. The lowest dose of SARS-CoV-2 RBD-Fc recombinant protein to trigger immunogenicity in monkeys was about 25 μg.

### EXAMPLE 11

### Detection of T-Cell Lymphocyte Responses in Collected Animal Sera Samples After Vaccination by IFN-γ ELISpot Assay

### Mouse T-Cell Lymphocyte Responses

Mouse splenocyte samples collected about 14 days after the 2^{nd} vaccination were crushed and isolated into single-cell suspension on 96-well nitrocellulose membrane plates. The single-cell suspension was cultured in R5 medium (RPMI 1640 with about 100 U/mL penicillin, about 100 U/mL streptomycin, about 5% heat-inactivated fetal bovine serum (FBS, Gibco, USA) and 2-mercaptoethanol). The cultured cells were harvested by transferring in a harvested tube and centrifuged at about 1,200 g, under a temperature of about 4° C. for about 5 minutes, then 1× ACK lysis buffer and R5 medium were added into the harvested tube. Centrifugation at about 1,200 g, under a temperature of about 4° C. for about 5 minutes was performed repeatedly. Splenocytes were obtained, collected, and subjected to analyzed IFN-γ titer using IFN-γ ELISpot assay. The splenocytes were diluted in R5 medium to obtain a cell concentration of about 5×10⁶ cells/ml, followed by incubation in 96-well nitrocellulose membrane plates (Millipore, Bedford, MA, USA) containing anti-mouse IFN-γ (AN18) monoclonal antibody (mAb) (Mabtech, Stockholm, Sweden) under a temperature of about 37° C., about 5% CO₂, for about 3 hours. The 96-well nitrocellulose membrane plates were washed about 6 times with 1× PBS before adding R10 medium (RPMI 1640 with about 100 U/mL penicillin, about 100 U/mL streptomycin, about 10% heat-inactivated fetal bovine serum (FBS, Gibco, USA) and 2-mercaptoethanol), incubated at a temperature of about 25 to about 30° C. for about 1 hour. About 2 μg/ml of SARS-CoV-2 (BioNet-Asia, Thailand, and Mimotopes, Australia) was added to the 96-well nitrocellulose membrane plates and incubated under a temperature of about 37° C., about 5% CO₂, for about 40 hours. The 96-well nitrocellulose membrane plates were washed about 6 times with 1× PBS and anti-mouse IFN-γ-biotinylated mAb (Mabtech,Stockholm, Sweden) was incubated under a temperature of about 37° C., for about 3 hours, followed by adding streptavidin-alkaline phosphatase (ALP: Mabtech, Stockholm, Sweden) and left for about 1 hour under a temperature of about 25 to about 30° C. About 1 μl substrate solution (5-bromo-4-chloro-3-indolyl-phosphate/nitro blue tetrazolium; BCIP/NBT) was added and the reaction was stopped by deionized water. ELISpot reader (ImmunoSpotÒ Analyzer, USA) and GraphPad Prism version 6.0 were used for analysis.

The results showed that SARS-CoV-2 RBD-Fc recombinant protein of the present invention exhibited an ability to significantly trigger mouse IFN-γ produced from T-cell lymphocyte compared to control group. Further, the results also showed that the production of mouse IFN-γ could be triggered even by immunizing with only the SARS-CoV-2 RBD-Fc protein.

### Monkey T-Cell Lymphocyte Responses

Peripheral blood mononuclear cell (PBMC) samples collected at day 14 after the 2^{nd} vaccination were prepared. Ethylene diamine tetraacetic acid (EDTA) was added in collected monkey blood, diluted in RPMI 1640 containing about 2 mM of L-Glutamine (Gibco, USA). The collected cells were centrifuged at about 1,200 g, under a temperature of about 4° C. for about 30 minutes, rinsed and washed with RPMI 1640 about two times. The collected cells were rinsed and washed again with R10 (RPMI 1640 with about 100 U/mL penicillin, about 100 U/mL streptomycin, about 10% heat-inactivated fetal bovine serum (FBS, Gibco, USA)) and subjected to analyze IFN-γ titer using IFN-γ ELISpot assay. On the 96-well nitrocellulose membrane plates, the collected cells were incubated with SARS-CoV-2 peptides (BioNet-Asia, Thailand, and Mimotopes, Australia) under a temperature of about 37° C., about 5% CO₂, for about 40 hours. About 50 μl of about 10 μg/ml of anti-monkey IFN-g-biotinylated mAb (Mabtech, Stockholm, Sweden) and about 100 μl (at about 1:200 diluted in PBS, about 0.5% FBS) of ALP solution (Mabtech, Stockholm, Sweden) were added to each well, then left for about 2 hours under a temperature of about 25 to about 30° C. About 100 μl substrate solution (5-bromo-4-chloro-3-indolyl-phosphate/nitro blue tetrazolium; BCIP/NBT) was added and the reaction was stopped by DI water. ELISpot reader (ImmunoSpotÒ Analyzer, USA) and GraphPad Prism version 6.0 were used for analysis.

The results showed that SARS-CoV-2 RBD-Fc recombinant protein in the present invention exhibited an ability to significantly trigger monkey IFN-γ produced from T-cell lymphocyte compared to the control group. Further, the results also showed that the production of mouse IFN-γ could be triggered by immunizing with SARS-CoV-2 RBD-Fc recombinant protein.

### EXAMPLE 12

### Stability of SARS-CoV-2 RBD-Fc Recombinant Protein for Long Term Storage

Further, the SARS-CoV-2 RBD-Fc recombinant protein was formulated with several excipients to determine the stability of long term storage. The optimal conditions for storing SARS-CoV-2 RBD-Fc recombinant protein throughout the study period for large-scale processing was investigated. In this study, excipients from various classes of stabilizers were selected in order to determine optimal conditions that can prolong the stability of SARS-CoV-2 RBD-Fc recombinant protein. The excipients comprising; 1) amino acid: glycine: 2) sugars: trehalose, sucrose, dextrose and sorbitol; and 3) surfactants: Tween-20 (polysorbate 20) and Tween-80 (polysorbate 80)

Preformulations thereof were formulated in PBS as follow,
Preformulation 1: 5% w/v trehalose;
Preformulation 2: 5% w/v trehalose+3% w/v glycine;
Preformulation 3: 5% w/v trehalose+5% w/v sorbitol;
Preformulation 4: 5% w/v trehalose+0.01% v/v Tween 20;
Preformulation 5: 5% w/v trehalose+0.01% v/v Tween 80;
Preformulation 6: 5% w/v sucrose+3% w/v glycine;
Preformulation 7: 5% w/v sucrose+5% w/v sorbitol; and
Preformulation 8: 5% w/v dextran+0.01% v/v Tween-80;
whereas, PBS was used as a control.

Then, different amount of SARS-CoV-2 RBD-Fc recombinant protein were investigated. Firstly, about 40 μg SARS-CoV-2 RBD-Fc recombinant protein was combined with eight groups of the preformulationsabove and stored under a temperature of about 2-8° C. for about 120 days. After 120 days, the protein in each group of the preformulations was determined by SDS-PAGE under reducing conditions. Testing results are shown in FIG. 12A. Moreover, about 5 μg SARS-CoV-2 RBD-Fc recombinant protein was also studied under the same conditions. Testing results are shown in FIG. 12B.

The results showed that both amounts of SARS-CoV-2 RBD-Fc recombinant protein at 40 μg and 5 μg in the preformulations2: 5% w/v trehalose+3% w/v glycine and those in the preformulations 6: 5% w/v sucrose +3% w/v glycine showed the highest stability among other formulations when stored at 2-8° C. for 120 days.

According to FIG. 12, Coomassie staining of each group of the preformulation after storing for about 120 days under a temperature of about 2-8° C. was determined by SDS-PAGE under reducing conditions. The 40 μg (FIG. 12A) and 5 μg (FIG. 12B) of total protein at day 0 were loaded. Ten lanes represented each group of preformulation as follows: 1) 5% w/v trehalose, 2) 5% w/v trehalose+3% w/v glycine, 3) 5% w/v trehalose+5% w/v sorbitol, 4) 5% w/v trehalose+0.01% v/v Tween 20, 5) 5% w/v trehalose+0.01% v/v Tween 80, 6) 5% w/v sucrose+3% w/v glycine, 7) 5% w/v sucrose+5% w/v sorbitol, 8) 5% w/v dextran+0.01% v/v Tween-80, 9) PBS and 10) PBS stored under a temperature of about −20° C. Protein ladder represented at left panel as kDa.

Based on the results, 5% w/v trehalose+3% w/v glycine (preformuation 2) and 5% w/v sucrose+3% w/v glycine (preformuation 6) played a significant role in the stability of plant-produced RBD-Fc for long-term storage conditions.

### EXAMPLE 13

### Representative Example of Vaccine Formulation for Administering to Mammals

The SARS-CoV-2 RBD-Fc recombinant protein in preformulation 6 (5% w/v sucrose and 3% w/v glycine in PBS) at the doses of about 5 μg and about 10 μg adjuvanted with aluminium hydroxide (Al(OH)₃, about 0.5 mg Al content), hereinafter referred to as SARS-CoV-2 RBD-Fc recombinant protein formulations, were administered to the cynomolgus monkeys as representative vaccine formulations.

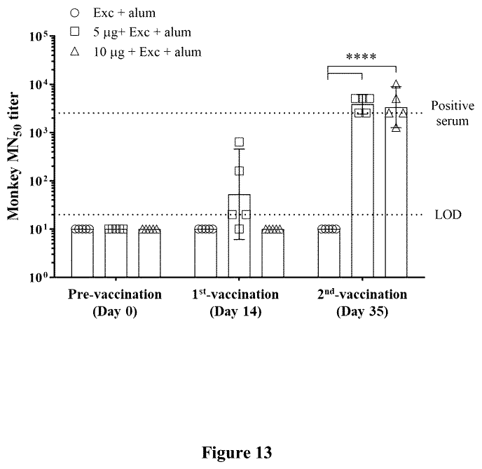

The in vitro neutralizing ability of the cynomolgus monkey immunized sera, which received two doses of the SARS-CoV-2 RBD-Fc recombinant protein formulations was shown in FIG. 13. The 15 cynomolgus monkeys were administered twice at Day 0 and Day 21 (21 day interval), then, the sera were collected at Day 0, Day 14 and Day 35 (14 days after vaccination). The immunized monkey sera were evaluated by microneutralization assay (MN). The neutralizing antibody level increased at Day 35 (14 days after the 2^{nd}-vaccination) in both 5 μg (geometric mean titer; GMT=3,380) and 10 μg (GMT=3,378) doses of SARS-CoV-2 RBD-Fc recombinant protein formulation with a significant difference when compared with the control group (excipients+alum), p-value<0.0001. From the results, SARS-CoV-2 RBD-Fc recombinant protein formulations showed dose-independent inducing neutralizing antibody against SARS-CoV-2 with no significant level between dose 5 and 10 μg. According to FIG. 13, The 50% microneutralizing titer (MN₅₀) of SARS-CoV-2 RBD-Fc recombinant protein cynomolgus monkey immunized sera were depicted. The 5 μg and 10 μg of SARS-CoV-2 RBD-Fc recombinant protein in excipients (Exc) adjuvanted with alum (5 μg+Exc+alum and 10 μg+Exc+alum) and in excipients adjuvanted with alum (Exc+alum) as a control group were injected to 15 cynomolgus monkeys. Monkey immunized sera were collected at Day 0 (pre-vaccination), Day 14 (14 days after 1^{st}-vaccination) and Day 35 (14 days after 2^{nd}-vaccination). Data presented as geometric mean±95% CI of the MN₅₀ titer in each group (n=5). Values smaller than the limit of detection (LOD) are plotted as 0.5*LOD. Two-way ANOVA, Tukey test, was used (****: p value<0.0001).

The results suggested that even at lower doses, both 5 μg and 10 μg of SARS-CoV-2 RBD-Fc recombinant protein combining with excipients (Exc) and adjuvant still demonstrated notable ability to trigger immunogenicity in monkeys. The results therefore implied that SARS-CoV-2 RBD-Fc recombinant protein formulations above were suitable for administering to mammals as representative formulations for vaccination.

Based on the results, SARS-CoV-2 RBD-Fc recombinant protein formulations as decribed above exhibited a strong ability as a vaccine for the prevention of the disease caused by severe acute respiratory syndrome coronavirus 2 (SARS-CoV-2).

As a person skilled in the art will readily appreciate, the above description is meant as an illustration of implementation of the principles of this invention. This description is not intended to limit the scope or application of this invention in that the invention is susceptible to modification, variation, and change, without departing from the spirit of this invention, as defined in the following claims.

### BEST MODE OF THE INVENTION

Best mode of the invention is as provided in the description of the invention.

1. A recombinant vector for producing immunogenic substance from plants comprising;
a) a severe acute respiratory syndrome coronavirus 2 surface glycoprotein sequence comprising amino acids 318-617 of SARS-CoV-2 receptor binding domain (SARS-CoV-2 RBD) protein sequence (SEQ ID NO: 2);
b) a fusion protein sequence which is fragment crystallizable (Fc) region from human immunoglobulin G1 (IgG1) (SEQ ID NO:6); and
c) a plant expression vector

2. The recombinant vector according to claim 1, wherein SARS-CoV-2 RBD protein sequence further comprises a signaling peptide (SEQ ID NO: 3) at N-terminus.

3. The recombinant vector according to claim 1, wherein SARS-CoV-2 RBD protein sequence further comprises a peptide linker at C-terminus for connecting SARS-CoV-2 RBD protein sequence to the fusion protein sequence (Fc).

4. The recombinant vector according to claim 3, wherein the peptide linker comprises 1 to 5 tandem repeats of (GGGGS)n (SEQ ID NO: 11).

5. The recombinant vector according to claim 4, wherein the peptide linker is (GGGGS)3 (SEQ ID NO: 5).

6. The recombinant vector according to claim 1, wherein the fusion protein sequence further comprises an endoplasmic reticulum retention motif at C-terminus.

7. The recombinant vector according to claim 6, wherein the endoplasmic reticulum retention motif is a SEKDEL motif (SEQ ID NO: 8).

8. The recombinant vector according to claim 1, wherein the plant expression vector derives from the bean yellow dwarf virus.

9. Host cell transformed, infected, or induced with the recombinant vector according to claim 1, wherein the host cell is a bacterial cell.

10. The host cell according to claim 9, wherein the host cell is Agrobacterium tumefaciens.

11. A plant cell infiltrated, transformed, infected, or induced with the host cell according to claim 10.

12. The plant cell according to claim 11, wherein the plant cell is Nicotiana sp.

13. The plant cell according to claim 12, wherein the plant cell is Nicotiana benthamiana.

14. A recombinant protein produced from the plant cell according to claim 13.

15. The recombinant protein according to claim 14, wherein the recombinant protein is severe acute respiratory syndrome-related coronavirus (SARSr-CoV) antigen.

16. The recombinant protein according to claim 15, wherein the severe acute respiratory syndrome-related coronavirus (SARSr-CoV) antigen is severe acute respiratory syndrome coronavirus 2 (SARS-CoV-2) antigen.

17. A use of the recombinant protein according to claim 14 in the manufacture of a medicament for the prevention of the disease caused by severe acute respiratory syndrome-related coronavirus (SARSr-CoV).

18. The use of the recombinant protein according to claim 17 in the manufacture of a medicament for the prevention of the disease caused by severe acute respiratory syndrome coronavirus 2 (SARS-CoV-2).

19. A method of inducing a protective immune response against SARS-CoV-2 antigen comprising administering the recombinant protein according to claim 14 to mammals in need thereof, wherein the recombinant protein induces a protective immune response against challenge with SARS-CoV-2 in mammals.

20. The method of inducing a protective immune response according to claim 19, wherein the recombinant protein is prepared as an intramuscular injection.

21. The method of inducing a protective immune response according to claim 19, wherein the administering step comprises at least two vaccinations.

22. The method of inducing a protective immune response according to claim 21, wherein the administering step comprises two vaccinations.

23. The method of inducing a protective immune response according to claim 22, wherein the second vaccination is administered 14-28 days after the first vaccination.

24. The method of inducing a protective immune response according to claim 23, wherein the second vaccination is administered 21 days after the first vaccination.


SEQUENCE LISTING (11 sequences)

SEQ ID NO: 1 - protein, 1273 aa, Severe acute respiratory syndrome coronavirus 2
  MISC_FEATURE (1)..(1273): surface glycoprotein [Severe acute respiratory syndrome coronavirus 2]
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 300 aa, Severe acute respiratory syndrome coronavirus 2
  MISC_FEATURE (1)..(300): surface glycoprotein [Severe acute respiratory syndrome coronavirus 2]
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNC

SEQ ID NO: 3 - protein, 25 aa, Nicotiana benthamiana
MGWSCIILFLVATATGVHSDVQLLE

SEQ ID NO: 4 - protein, 333 aa, Nicotiana benthamiana
MGWSCIILFLVATATGVHSDVQLLEFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCDISGGGGS

SEQ ID NO: 5 - protein, 15 aa, Artificial Sequence
  Synthetic linker
GGGGSGGGGSGGGGS

SEQ ID NO: 6 - protein, 221 aa, Nicotiana benthamiana
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 564 aa, Nicotiana benthamiana
MGWSCIILFLVATATGVHSDVQLLEFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCDISGGGGSGGGGSGGGGSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 6 aa, Nicotiana benthamiana
SEKDEL

SEQ ID NO: 9 - protein, 239 aa, Nicotiana benthamiana
GSGGGGSGGGGSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 10 - protein, 570 aa, Nicotiana benthamiana
MGWSCIILFLVATATGVHSDVQLLEFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCDISGGGGSGGGGSGGGGSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 11 - protein, 25 aa, Artificial Sequence
  Synthetic linker
  misc_feature (1)..(25): This sequence may encompass 1-5 “Gly Gly Gly Gly Ser” repeating units
GGGGSGGGGSGGGGSGGGGSGGGGS